(19) United States
(12) Patent Application Publication
(10) Pub. No.: US 2024/0298945 A1
(43) Pub. Date: Sep. 12, 2024

(54) SYSTEM AND METHOD FOR DETECTING AN EMOTIONAL STATE

(71) Applicant: Northwestern University, Evanston, IL (US)

(72) Inventors: Jeffrey S. Burgdorf, Evanston, IL (US); Joseph Moskal, Evanston, IL (US)

(73) Assignee: Northwestern University, Evanston, IL (US)

(21) Appl. No.: 18/597,748

(22) Filed: Mar. 6, 2024

Related U.S. Application Data
(60) Provisional application No. 63/450,502, filed on Mar. 7, 2023.

(51) Int. Cl. A61B 5/16; A61B 5/00; A61B 5/11; A61B 5/389; G06F 1/16; G08B 21/18

(52) U.S. Cl. CPC A61B 5/165; A61B 5/0006; A61B 5/11; A61B 5/389; G06F 1/163; G08B 21/182

(57) ABSTRACT

The present disclosure is generally directed to a system and method for monitoring and treating an emotional state. The system includes a device including an electroencephalogram, an electromyography, a sound sensor, an accelerometer, and a gyroscope. The device is communicably coupled to a converter configured to convert the data and send the data to an external device. The external device is configured to store and analyze the data. The external device in configured to send a signal to perform a medical intervention.

### CROSS-REFERENCE TO RELATED APPLICATION

This application claims the benefit of U.S. Provisional Patent Application No. 63/450,502, filed Mar. 7, 2023, which is incorporated by reference herein in its entirety.

### TECHNICAL FIELD

The present disclosure relates generally to an apparatus, system, and method for monitoring and treating an emotional state. More specifically, the present invention relates to predicting an emotional state to provide an intervention.

### BACKGROUND

The following discussion is provided to aid the reader in understanding the disclosure and is not admitted to describe or constitute prior art thereto.

Mental health is a key part to everyday life. In recent years, mental health awareness has risen, however, many people lack the resources and medical intervention needed to manage mental illness. Mental and emotional states can change quickly and may be affected by a variety of factors within a patient's life. An event that triggers an emotional state can happen over long periods of time or over a matter of seconds. Thus, emotional states can be challenging to assess and provide medical intervention. It is critical that medical intervention occurs prior to an adverse event occurring.

Psychiatric and mental health evaluations often occur in a medical setting. Sometimes an evaluation may occur in an outpatient setting in which the healthcare provider (e.g., physician) may only have a set amount of time (e.g., an appointment window) to evaluate the patient's mental state. The patient may be reluctant to share insight into how they are feeling mentally and emotionally.

Many patients are treated for psychiatric disorders in an inpatient setting. An inpatient setting can add additional stress and emotional burden to the patient by taking them out of their usual environment. Admitting patients into an inpatient setting may be the best option for their personal safety and for administering medical interventions, however, the mental and emotional toll of being outside of their home environment may impose irreversible implications. For example, a patient may become accustomed to life in an inpatient care setting. For example, the patient may become accustomed to being monitored more closely and having the stress of everyday tasks and decisions alleviated. Therefore, when the patient returns to their normal setting, without the support of inpatient care healthcare providers, they may be more susceptible to a relapse. A relapse may correlate to a variety of adverse events including, but not limited to, self-harm, suicide, and substance abuse. Furthermore, in an inpatient setting, the healthcare provider has multiple patients to monitor and treat simultaneously. Therefore, the healthcare provider may not be able to monitor each patient at any given time, making it difficult to predict a shift in emotional state and provide an intervention to the patient before an adverse event may occur.

A patient's emotional state may also change based on their setting and surrounding stimuli. For example, a patient may present stable and well in a clinical setting, while they may quickly turn to unstable and unpredictable when facing a stressful situation (e.g., job stress, social stress, financial stress, etc.). Unfortunately, a physician may not be present when a shift in emotional state occurs, thus they may not be able to provide a medical intervention prior to an adverse event occurring.

Therefore, the deficiencies herein described have left a critical gap in patient care which ultimately may result in an adverse event that may otherwise be preventable. There is a need for a system to continuously monitor and evaluate the emotional state of a patient. Furthermore, there is a need for a system that does not disrupt the patient's everyday life. A device that passively monitors multiple biosignals to evaluate the emotional state of a patient is critical in providing the best care to the patient without interrupting daily life. This is important because interruptions to a patient's life may be a triggering event which may lead to a shift in emotional state or an adverse event.

Varying populations are in need of continuous, point of care, medical treatment to monitor and treat emotional states correlated to a variety of illnesses. There is a need for a biosensor and algorithm that can predict the onset of negative emotional expression in time to provide an appropriate intervention. For example, the medical intervention may prevent suicide or addiction relapse.

Specifically, there is a need for monitoring an emotional state of patient presenting suicidal thoughts and actions. Suicide rates are rising, and although there are a variety of interventions available to treat suicidal thoughts and actions, many are not receiving proper treatment. Patients may avoid treatment for various reasons. For example, a patient may avoid seeking help due to the perceived stigma around mental health. They may also avoid treatment due to the fear of inpatient care or interruption to their everyday life. An interruption to their everyday life may force them to share personal details of their emotional state and treatment. Thus, there is a need to monitor emotional states and provide a medical intervention to patients outside of a healthcare setting to prevent self-harm and suicidal actions.

There is also a need for a monitoring an emotional state of patients dealing with substance abuse, such as drug and/or alcohol abuse. For example, there is a need for a device and method for monitoring chemical change in the brain as well as physiological changes (e.g., heart rate) that may be indicative of a relapse event occurring in the future. By predicting an emotion state, such as a craving for a substance, a medical intervention can be provided prior to the patient acting to satisfy the craving. By providing a medical intervention, such as a medication, or support, to prevent a relapse in substance abuse, the physician may be able to aid the patient through stages of sobriety thus increasing the patient's quality of life.

Another population in need of monitoring an emotional state in order to provide a medical intervention, is patient's suffering from mental deterioration, such as Alzheimer's or dementia. Patient's suffering from Alzheimer's may experience confusion, irritability, agitation, or other shifts in emotional state in the afternoon or early evening (e.g., sundowning, etc.). Medical intervention such as providing medications (e.g., antipsychotics, etc.), or providing mental tasks may prevent sundowning, or confusion, from occurring.

### SUMMARY

This disclosure relates generally to an embodiment for monitoring a mental state based on multiple biosignals and performing a calculation on said biosignals to predict a mental or emotional state and provide a medical intervention. Furthermore, an example of this embodiment is a method for monitoring an emotional state of a suicidal patient and providing medical intervention. Another example of this embodiment is a method for monitoring an emotional state of a patient recovering from addiction and providing a medical intervention.

One embodiment relates to a device for detecting an emotional state. The device includes an electroencephalogram (EEG) configured to measure electrical activity in a brain of a patient. The EEG includes a plurality of EEG biosensors. The device also includes an electromyogram (EMG) configured to measure electrical activity in a muscle and nerve of a patient. The EMG includes a plurality of EMG biosensors. The device further includes a sound sensor configured to detect sound in the patient's environment, an accelerometer configured to detect the frequency and intensity of the patient's movement over time, and a gyroscope configured to detect rotational movement and orientation of the patient over time.

Another embodiment relates to a system for detecting an emotional state. The system includes a wearable device for detecting an emotional state. The wearable device includes an electroencephalogram (EEG) configured to measure electrical activity in a brain of a patient. The EEG includes a plurality of EEG biosensors. The wearable device includes an electromyogram (EMG) configured to measure electrical activity in a muscle and nerve of a patient. The EMG includes a plurality of EMG biosensors. The wearable device further includes a sound sensor configured to detect sound in the patient's environment, an accelerometer configured to detect the frequency and intensity of the patient's movement over time, and a gyroscope configured to detect rotational movement and orientation of the patient over time. The system also includes a converter communicably coupled to the wearable device. The converter configured to receive a plurality of biosensor data from the wearable device, and convert the plurality of biosensor data to a plurality of digital data. The system further includes an external device comprising a computer readable storage media and a processor. The external device is configured to receive the digital data from the converter, store the digital data in a cloud drive, analyze the digital data, determine if a signal is needed, and send a signal to the patient indicating a medical intervention.

Another embodiment is related to a method for detecting an emotional state. The method includes collecting, on a device, a plurality of biosensor data. The device is configured to determine, by an electroencephalogram (EEG), electrical activity of a patient's brain, determine, by an electromyogram (EMG), electrical activity of the patient's muscle and nerve, detect, by a sound sensor, a sound in the patient's environment, detect, by an accelerometer, the frequency and intensity of the patient's movement over time, and detect, by a gyroscope, rotational movement and orientation of the patient over time. The method also includes converting, by a converter, the plurality of biosensor data to a plurality of digital data, and sending the plurality of digital data to an external device. The external device includes a computer readable storage media and a processor. The external device is configured to receive the digital data from the converter, store the digital data in a cloud drive, analyze the digital data, determine if a signal is needed, and send a signal to the patient indicating a medical intervention.

### BRIEF DESCRIPTION OF THE DRAWINGS

FIG. 1 is a block diagram of a device for monitoring and treating an emotional state of a patient.

FIG. 2 is a flow chart of a system including the device of FIG. 1 for monitoring and treating an emotional state of a patient.

FIGS. 3A-3C illustrate experimental data relating to treating an emotional state of a patient as illustrated in the system of FIG. 2.

FIG. 4 illustrates experimental data relating to treating an emotional state of a patient by detecting a shift in EEG data as illustrated in the system of FIG. 2.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

Unless the context indicates otherwise, it is specifically intended that the various features of the invention described herein can be used in any combination. Moreover, the disclosure also contemplates that in one or more embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

### Definitions

The expression “comprising” means “including, but not limited to.”

As used herein, the term “subject” is used interchangeably with “patient” and is referring to a human.

A “medical intervention” is administering any action with the intention of preventing an adverse medical event.

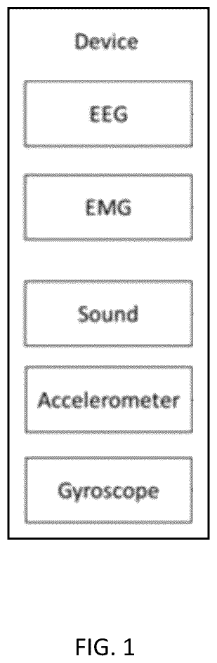

An “effective amount” is an amount sufficient to effect beneficial or desired results. An effective amount can be administered in one or more administrations, applications or dosages based on patient specific metrics. The following method may refer generally to the exemplary embodiments of FIGS. 1-3C.

### Methods

The present disclosure is directed to a novel device and method for continuously monitoring an emotional state of a patient such that medical intervention can be initiated in a point of care setting to prevent an adverse event.

Referring generally to the FIGURES, is a device and method for monitoring and treating an emotional state of a patient.

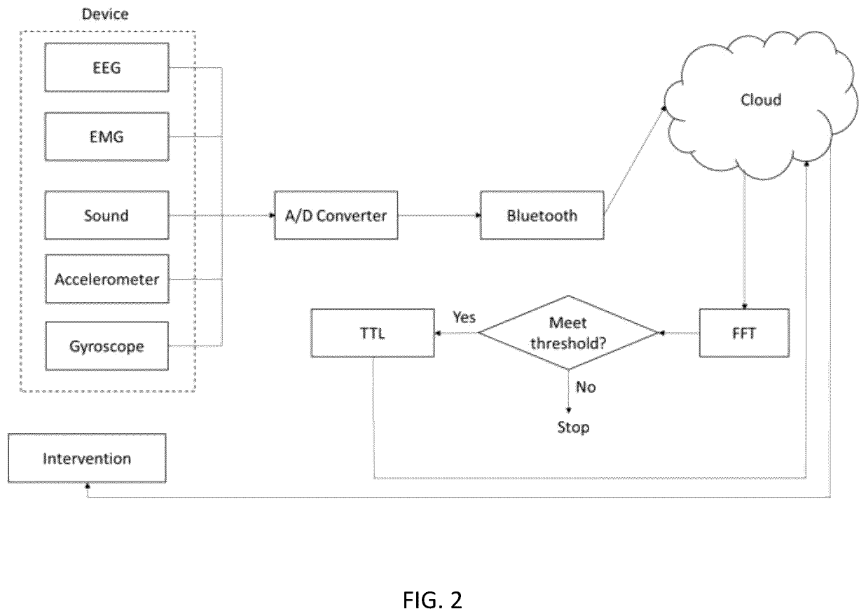

Referring generally to FIG. 1 and FIG. 2, is a device and system for monitoring and treating an emotional state of a patient. Specifically, FIG. 2 is a flow chart illustrating a system for monitoring an emotional state of a patient. The system includes a device configured to monitor various signals and send said signals to a cloud serves. The device may be a wearable device. Furthermore, the device may be a communicably coupled to an external device such as a computer or database. The device includes a plurality of biosensors. The biosensors are configured to monitor a plurality of biosignals. For example, the device includes an electroencephalogram (EEG). The electroencephalogram (EEG) is configured to measure electrical activity in the brain. For example, spikes in activity in certain areas of the brain may be correlated to certain emotions. The EEG device includes a plurality of EEG sensors. The EEG sensors may be placed on the patient's head and transfer the measured electrical activity of the brain to the device via a wired connection or via a wireless Bluetooth connection. Instead, in some embodiments, the EEG may transfer the measured electrical activity of the brain directly to the external computer via Bluetooth. The EEG device may also be configured to deliver electrical pulses to the patient's scalp as a medical intervention.

Furthermore, the device includes an electromyography (EMG) device. The EMG device is configured to measure electrical activity in nerves and muscles. For example, activation and/or repeated activation of specific nerves and muscles may be correlated with another emotional state. The EMG device includes a plurality of EMG sensors. The EMG sensors may send the measured electrical activity of the muscle of interest to the wearable device via a wired connection or via Bluetooth connection. The plurality of EMG sensors may be placed near any muscle of interest on the patient's body. For example, the EMG sensors may be placed on the patient's neck. By placing the EMG sensors on the neck, the EMG device may sense elevated muscular activity (e.g., tension, etc.) in the neck during a change in emotional state. For example, stress may induce tension in muscles, such as the neck. When tension is sensed in the targeted muscle, the patient may be experiencing a shift in emotional state. For example, the patient may be shifting from a positive, relaxed state, to a stressed, negative state. The shift in emotional state may trigger an adverse event, such as suicidal thoughts or actions, or a lapse in judgement to revert to substance abuse. The EMG device may further be configured to deliver an electrical pulse to the patient's muscle as a medical intervention.

The device may further include a sound detection device. The sound detection device may be configured to detect loud alarming sounds that may be indicative of a stressful atmosphere. For example, the sound detection device may detect an emotional state by sensing yelling or crying indicative of an emotional shift. The sound detection along with the EEG measured brain activity and the EMG measured muscle activity may be used to collectively monitor and predict and emotional state. For example, the combination of a loud environment, along with brain activity in certain parts of the brain (e.g., parts associated with suicidal thoughts and ideation, etc.), and activation of muscles indicative of stress, may indicate a shift in an emotional state. For example, the combination of multiple signals indicative of a shift in emotional state may indicate a strong shift such that a different mode of intervention is provided.

Furthermore, the device includes an accelerometer and a gyroscope. The accelerometer is configured to detect motion. The gyroscope is configured to detect the orientation of the device as it moves with the patient (e.g., the user). For example, the accelerometer and gyroscope may detect erratic movement that may be correlated to a shift in emotional state. For example, the wearable device may be placed on the patient's head (e.g., a headband, a hat, etc.) such that the accelerometer and gyroscope can detect the patient's movement as well as the orientation of their head. For example, when a shift in emotional state occurs, the patient may start moving faster or rotating their head repeatedly. In other embodiments, the wearable device may be positioned on the patient's arm. For example, the wearable device may be incorporated into a watch or bracelet.

In another embodiment, the wearable device may be positioned near the patient's waist. For example, the device may be clipped onto the patient's clothing (e.g., a belt, waistband, etc.) and then communicate with biosensors placed on the patient's body. For example, the patient may have sensors placed near their head, neck, and limbs that communicates with a central wearable device that collects the data. The biosensors may communicate with the wearable device via a wire connection or through a wireless connection (e.g., Bluetooth, etc.). In yet another embodiment, the patient may wear biosensors (e.g., wireless sensors, sensors with a strap, stick on sensors, etc.) on various parts of their body that can communicate with a device proximate to the patient. For example, the device may be a watch (e.g., a smartwatch, etc.), a phone (e.g., a smartphone, etc.) a computer (e.g., a hard drive, a laptop, a tablet, a desktop, etc.), or any other device including communication capabilities (e.g., Bluetooth connection, storage capability, Wi-Fi connection, etc.).

Once the data is collected from the EEG, the EMG, the sound device, the accelerometer, and the gyroscope, the device digitizes the biosensor data. For example, the biosensor data may be digitized by an analog-to-digital converter (e.g., an A/D converter) or another converter device. For example, the A/D converter may convert the analog current signals of the EEG and the EMG to a digital number that represents the measured current. The A/D converter may also convert the detected sound signal to a digital signal. The A/D converter is configured to convert continuous signals to discrete signals.

After the biosensor data is digitized, the biosensor data is sent to the external device via Bluetooth. The external device may be a computer, or another device with a memory unit and processor. The external device may include a cloud drive. The cloud drive is configured to store the digitized biosensor data such that the digitized data may be analyzed or utilized collectively or at a later time.

Once the data is digitized and stored in the cloud drive, the external device is then configured to run an algorithm. The algorithm may be configured to transform the biosensor data. For example, the algorithm may perform a Fast Fourier Transformation (FFT). For example, a FFT may be created for each of the biosignals within the cloud drive to create individual power bands. The external device is also configured to calculate the power and coherence within each individual power band from each FFT. Furthermore, stored within the external device is a threshold for each of the biosignals. The thresholds may be predetermined by a physician. The external device is further configured to determine if each biosignal meets or exceeds its corresponding threshold. If a biosignal meets or exceeds the threshold, a signal is then sent from the external device to deliver a medical intervention. The medical intervention may consist of but is not limited to, delivery of a drug (e.g., an effective amount, etc.), delivery of an electrical or magnetic stimulation, and/or contacting a healthcare provider.

In some embodiments, if a biosignal meets or exceeds the threshold, a signal is sent directly to the patient. For example, the signal may be sent to the patient through the wearable device or through a mobile device. The signal may direct the patient to take a medication (e.g., self-administer a drug). Furthermore, once the patient has taken the medication, the patient may be prompted to acknowledge that they have completed the task of taking the medication. For example, the patient may press a button to record completion or record a verbal acknowledgement over a phone call. In some embodiments, after completing the administered medical intervention, the patient may be prompted to take further action, such as contacting a physician or family member, or scheduling an appointment with a healthcare provider.

In another embodiment, the wearable device may be configured to directly deliver a drug to the patient. For example, the wearable device may include a port that subcutaneously delivers a drug to a patient. The device may house an effective dose of the medication and inject the dose into the patient when at least one biosignal reaches or exceeds a threshold. Furthermore, this embodiment may be configured to notify a healthcare provider or specific caregiver that a medical intervention has been administered.

The system of FIG. 2 is advantageous for providing continuous monitoring of a patient. Specifically, the system is advantageous in an outpatient setting. For example, a patient may be able to return home while still receiving continuous monitoring and care. Furthermore, the system may allow a healthcare provider (e.g., physician, nurse, etc.) to monitor multiple patients simultaneously. For example, multiple devices may be configured to send the biosensor data to a singular computer or database such that a heath care provider can view data from multiple patients. This may be especially advantageous during the early morning or nighttime when less healthcare workers may be available. This invention also increases the efficiency of healthcare providers caring for patients with various mental illnesses.

### Experimental Data

### Experiment 1

Experiments were approved by the Northwestern University (Evanston IL), or Northshore Hospital (Evanston IL) IACUC committees and were carried out in accordance with either the Guide for the Care and Use of Laboratory Animals as adopted and promulgated by the U.S. National Institutes of Health.

In one experiment, Male 2- to 3-month-old Sprague-Dawley (SD) rats from Charles River (United States) were used. Rats were housed in Lucite cages with aspen-wood chip bedding, maintained on a 12:12 light:dark cycle (lights-on at 6 am), and given ad libitum access to Teklad lab chow (Envigo, United States) and tap water throughout the study.

In the discussed experiments, rats were anesthetized with isoflurane (5% induction and 2-3% maintenance; 15-20 min total duration) and implanted with skull screws to record cortical EEG (Pinnacle, USA), as well as depth medial prefrontal cortex (MPFC) and periaqueductal gray (PAG) recording and stimulating electrodes. The animals were given 7 days to recover before the start of testing. Auditory event-related potentials (ERPs) were recorded from a frontal cortex skull screw using a cerebellar skull screw as a ground/reference. EEG signals were captured via a tethered system (Pinnacle, USA). Data were acquired at 10 kHz using an A&M (USA) amplifier with a high (0.1 Hz)- and low-pass (100 Hz) filters and digitized using Data Wave (USA) acquisition software. Data were analyzed using Brain Products Analyzer 2 software (Germany).

During the closed loop stimulation studies, real time alpha and delta ratios were calculated in 30 second intervals using a custom data wave script (A-M systems, USA). Initial baseline recordings were obtained to determine the threshold for closed loop stimulation for each animal (3 X SD of baseline alpha delta ratio). Theta train stimulation was delivered either once the threshold was reached or delivered at a yoked random control time point. Aversive ultrasonic vocalizations (aversive USVs) were induced either by aversive tickling (across a 3 min test session) or 23 hours of sleep deprivation by gentle handing (pinnacle, USA).

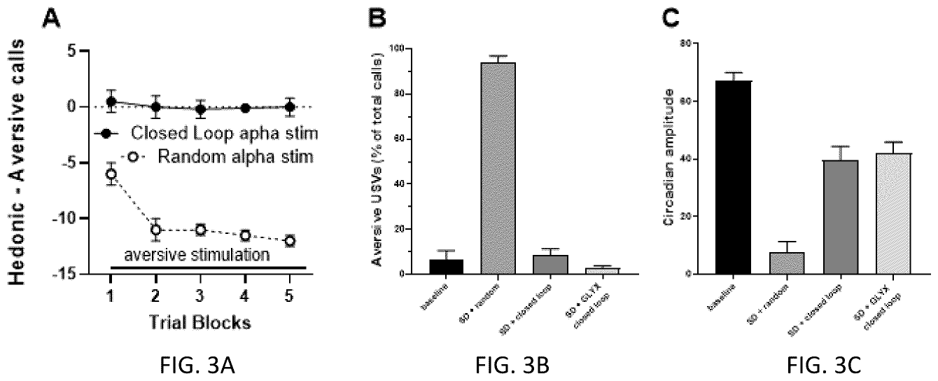

FIG. 3A, FIG. 3B, and FIG. 3C illustrate the effects of providing medical intervention in response to determining an emotional state. Various types of medication intervention may be delivered solely or in combination. For example, administering an electrical pulse and delivering a drug may be advantageous to the patient by themselves and in combination. The electrical pulse may be delivered to a patient's scalp through the EEG or to the patient's muscle through the EMG of the wearable to device to observe similar outcomes as those shown through FIGS. 3A-3C. In some embodiments a combination of electrical pulses sent by the EEG and EMG may be utilized during a medical intervention. For example, this may allow healthcare providers to deliver personalized care based on patient specific needs.

As shown in FIG. 3A, administering alpha stimulation during the start of change in an EEG (e.g., delta EEG, etc.) may prevent the transition from a positive to a negative emotional state during unpleasant stimuli. Specifically, the use of closed loop alpha stimulation (shown in solid black circles) prevents the transition from a positive state to a negative state. For example, the administration of closed loop alpha stimulation at the onset of changes in the biosensor data may prevent a patient from inflicting self-harm (e.g., suicide) due to prevention of transitioning from a positive or neutral emotional state to a negative emotional state.

Furthermore, FIG. 3B and FIG. 3C illustrate the effects of administering random treatment to a sleep deprived (SD) group, alpha stimulation to a sleep deprived group, and alpha stimulation and a drug to a sleep deprived group as compared to a baseline group. The aversive calls as a percent of total calls was the measurement tool used to determine effectiveness of each treatment. As shown in FIG. 3B, administering alpha stimulation (e.g., closed loop alpha stimulation, etc.) and a drug prevents aversive vocalization. For example, the group administered an antidepressant (e.g., GLYX-13, etc.) and closed loop alpha stimulation at the onset of change in EEG activity had the lowest percent of aversive, strong dislike or negative, calls out of total calls. The group administered only closed loop alpha stimulation at the onset of change in EEG activity had a lower percentage of negative calls than the group administered random alpha stimulation, but had a higher percentage of negative calls than the group administered both an antidepressant and closed loop alpha stimulation.

As shown in FIG. 3C, the use of alpha stimulation may further affect circadian rhythm (e.g., sleep, etc.). For example, a greater value of a circadian amplitude may indicate a healthy circadian rhythm. For example, patients experiencing emotional distress, or elderly patients, may have a lesser circadian amplitude may be a signal of a poor circadian rhythm. A healthy circadian rhythm is critical for mental and emotional health as well as physical health. The administration of an antidepressant, such as GLYX-13 and closed loop alpha stimulation at the onset of change in EEG may restore the patient's circadian amplitude to higher levels. The group administered both an antidepressant and closed loop alpha stimulation had significantly higher circadian amplitudes than the group administered random alpha stimulation. For example, the use of antidepressants and closed loop alpha stimulation in suicidal patients at the onset of change in their EEG may prevent the shift to a negative emotional state as well as help restore a normal circadian rhythm and sleep cycle which may also contribute to improving mental health. In other patients, such as patients suffering from Alzheimer's, the use of alpha stimulation and/or an antidepressant may prevent the phenomena of sundowning (e.g., confusion, irritability, etc.).

### Experiment 2

In another analysis, the Nationwide Children's Hospital (NCH) sleep dataset (1) wake EEG data was analyzed with permission from the National Sleep Research Resource database (2). Quantitative EEG data from 42 recordings from 14 heathy control subjects and 24 age-matched subjects with suicide ideation were analyzed via brain vision analyzer (Germany). Normalized EEG power from the frontal electrodes (average of C3 and C4) were calculated and the alpha (12-13.5) delta (1.5-2 Hz) ratio (alpha/alpha+delta) was computed.

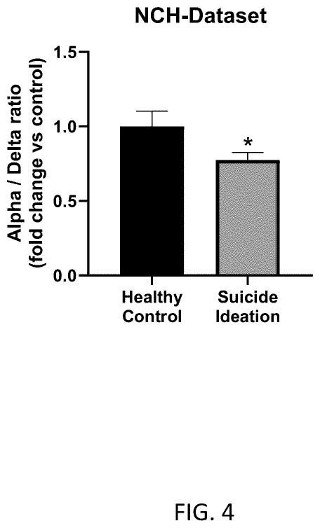

FIG. 4 illustrates the alpha delta ratio calculated from the quantitative EEG data received from both subject groups. As shown in FIG. 4, subjects with suicide ideation showed a lower alpha delta ratio compared to healthy controls (t(40)=2.173, p=0.0358 unpaired t-test two tailed).

By collecting quantitative EEG data on a patient over time, the patient's alpha delta ratio can be calculated and monitors. When a shift in the patient's alpha delta ratio is observed, treatment can be administered. For example, the use of alpha stimulation, such as closed loop alpha stimulation, can be administered following (e.g., in response to, etc.) the detection of a decrease in the patient's alpha delta ratio (e.g., below a threshold value, a predetermined value, etc.), to increase the patient's alpha delta ratio back to a baseline value or within an expected range. By providing a medical intervention (e.g., alpha stimulation, etc.), suicidal ideation may decrease, thus improving the mental health and overall well-being of the patient.

Further, in response to the detection of a shift (e.g., decrease, etc.) in a patient's alpha delta ratio, a medication, such as an antidepressant, and closed loop alpha stimulation may be administered. For example, by providing an antidepressant and alpha stimulation to a suicidal patient at the onset of change in their alpha delta ratio calculated from their EEG may prevent further decrease to their alpha delta ratio, thus improving the patient's mental health decreases instances of suicidal ideation.

While certain embodiments have been illustrated and described, it should be understood that changes and modifications can be made therein in accordance with ordinary skill in the art without departing from the invention in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed invention.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting. Any recited method can be carried out in the order of events recited or in any other order which is logically possible.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

### CITED REFERENCES

1. Lee H, Li B, DeForte S, Splaingard M L, Huang Y, Chi Y, Linwood S L. A large collection of real-world pediatric sleep studies. Sci Data. 2022 Jul. 19; 9(1):421. doi: 10.1038/s41597-022-01545-6. PMID: 35853958; PMCID: PMC9296671.
2. Zhang G Q, Cui L, Mueller R, Tao S, Kim M, Rueschman M, Mariani S, Mobley D, Redline S. The National Sleep Research Resource: towards a sleep data commons. J Am Med Inform Assoc. 2018 Oct. 1; 25(10):1351-1358. doi: 10.1093/jamia/ocy064. PMID: 29860441; PMCID: PMC6188513.

What is claimed is:

1. A device for detecting an emotional state, comprising:
an electroencephalogram (EEG) configured to measure electrical activity in a brain of a patient, the EEG comprising a plurality of EEG biosensors;
an electromyogram (EMG) configured to measure electrical activity in a muscle and nerve of a patient, the EMG comprising a plurality of EMG biosensors;
a sound sensor configured to detect sound in the patient's environment;
an accelerometer configured to detect the frequency and intensity of the patient's movement over time; and
a gyroscope configured to detect rotational movement and orientation of the patient over time.

2. The device of claim 1, wherein the device is a wearable device, communicably coupled an external device, the external device comprising a computer readable storage media and a processor.

3. The device of claim 1, wherein the device is further comprising a converter, the converter configured to:
receive a plurality of biosensor data from the wearable device, and
convert the plurality of biosensor data to a plurality of digital data.

4. The device of claim 1, wherein the device is further configured to transmit a signal to deliver a medical intervention.

5. The device of claim 1, wherein the device is a wearable device configured to be worn outside of a clinical setting and communicate with a health care provider.

6. A system for detecting an emotional state comprising:
a wearable device for detecting an emotional state, comprising:
an electroencephalogram (EEG) configured to measure electrical activity in a brain of a patient, the EEG comprising a plurality of EEG biosensors,
an electromyogram (EMG) configured to measure electrical activity in a muscle and nerve of a patient, the EMG comprising a plurality of EMG biosensors,
a sound sensor configured to detect sound in the patient's environment,
an accelerometer configured to detect the frequency and intensity of the patient's movement over time, and
a gyroscope configured to detect rotational movement and orientation of the patient over time;
a converter communicably coupled to the wearable device, the converter configured to:
receive a plurality of biosensor data from the wearable device, and
convert the plurality of biosensor data to a plurality of digital data; and
an external device comprising a computer readable storage media and a processor, the external device configured to:
receive the digital data from the converter,
store the digital data in a cloud drive,
analyze the digital data,
in response to analyzing the digital data, determine if a signal is needed, and
in response to determining a signal is needed, send a signal to the patient indicating a medical intervention.

7. The system of claim 6, wherein analyzing the digital data comprises:
performing an algorithm on the plurality of digital data;
determining if the digital data meets or exceeds a predefined threshold; and
in response to the digital data meeting or exceeding the predefined threshold, sending a signal to deliver an intervention.

8. The system of claim 7, wherein in response to determining the digital data does not meet or exceed the predefined threshold, the external device ends the analysis.

9. The system of claim 7, wherein the algorithm includes creating a Fast Fourier Transformation (FFT) on the plurality of digital data, wherein creating a Fast Fourier Transformation of the data creates a plurality of individual power bands.

10. The system of claim 7, wherein the algorithm further includes calculating a power and a coherence for each of the plurality of individual power bands.

11. The system of claim 6, wherein the external device is a computer, the computer configured to receive a plurality of digital data from a plurality of patients.

12. The system of claim 6, wherein the medical intervention comprises administering a drug.

13. The system of claim 6, wherein the medical intervention comprises administering an electrical or magnetic stimulation to the patient.

14. The system of claim 6, wherein the medical intervention comprises administering a drug and administering an electrical or magnetic stimulation to the patient.

15. The system of claim 6, wherein the external device is configured to notify a health care provider in response to the digital data meeting or exceeding the predefined threshold.

16. A method for detecting an emotional state comprising:
collecting, on a device, a plurality of biosensor data, the device configured to:
determine, by an electroencephalogram (EEG), electrical activity of a patient's brain,
determine, by an electromyogram (EMG), electrical activity of the patient's muscle and nerve,
detect, by a sound sensor, a sound in the patient's environment,
detect, by an accelerometer, the frequency and intensity of the patient's movement over time, and
detect, by a gyroscope, rotational movement and orientation of the patient over time;
converting, by a converter, the plurality of biosensor data to a plurality of digital data; and
sending the plurality of digital data to an external device comprising a computer readable storage media and a processor, the external device configured to:
receive the digital data from the converter,
store the digital data in a cloud drive,
analyze the digital data,
in response to analyzing the digital data, determine if a signal is needed, and
in response to determining a signal is needed, send a signal to the patient indicating a medical intervention.

17. The method of claim 6, wherein analyzing the digital data comprises:
performing an algorithm on the plurality of digital data;
determining if the digital data meets or exceeds a predefined threshold; and
in response to the digital data meeting or exceeding the predefined threshold, sending a signal to deliver an intervention.

18. The method of claim 17, wherein the algorithm includes creating a Fast Fourier Transformation (FFT) on the plurality of digital data, wherein creating a Fast Fourier Transformation of the data creates a plurality of individual power bands.

19. The system of claim 18, wherein the algorithm further includes calculating a power and a coherence for each of the plurality of individual power bands.

20. The system of claim 16, wherein the medical intervention comprises administering at least one of a drug, an electrical stimulation, and a magnetic stimulation.
